(19) United States
(12) Patent Application Publication
(10) Pub. No.: US 2025/0268960 A1
(43) Pub. Date: Aug. 28, 2025

(54) COMPOSITION FOR PREVENTION AND TREATMENT OF OBESITY OR INSULIN RESISTANCE SYNDROME COMPRISING LACTOBACILLUS PLANTARUM HAC03 AND RUTIN

(71) Applicant: HANDONG GLOBAL UNIVERSITY INDUSTRY-ACADEMIC COOPERATION FOUNDATION, Pohangsi Gyeongsangbuk-do (KR)

(72) Inventors: Jin Hwan KWAK, Pohang-si Gyeongsangbuk-do (KR); Ah Ram KIM, Pohang-si Gyeongsangbuk-do (KR); Youn Goo KANG, Pohang-si Gyeongsangbuk-do (KR)

(21) Appl. No.: 19/074,320

(22) Filed: Mar. 8, 2025

Related U.S. Application Data
(63) Continuation of application No. PCT/KR2024/013756, filed on Sep. 11, 2024.

(30) Foreign Application Priority Data
Sep. 21, 2023 (KR) 10-2023-0125983

(51) Int. Cl. A61K 35/747; A61K 9/00; A61K 31/7048; A61K 35/00; A61P 3/06; A61P 3/10

(52) U.S. Cl. CPC A61K 35/747; A61K 9/0056; A61K 31/7048; A61P 3/06; A61P 3/10; A61K 2035/115

(57) ABSTRACT

The present disclosure relates to a pharmaceutical composition and a health functional food composition for preventing or treating obesity or insulin resistance syndrome, containing Lactobacillus plantarum HAC03 strain and rutin as active ingredients. The combination of Lactobacillus plantarum HAC03 strain and rutin of the present disclosure has the efficacies of reducing body weight gain and tissue weight, reducing tissue cell size, inhibiting lipogenesis, and increasing fat oxidation. Additionally, the pharmaceutical composition has the efficacies of reducing blood sugar fluctuations and reducing fasting blood sugar levels and fasting insulin levels, and thus it can be used as a therapeutic agent for obesity or insulin resistance syndrome, a health functional food, etc.

### TECHNICAL FIELD

The present disclosure relates to a pharmaceutical composition and a health functional food composition for preventing or treating obesity or insulin resistance syndrome, containing Lactobacillus plantarum HAC03 strain and rutin as active ingredients.

### REFERENCE TO SEQUENCE LISTING SUBMITTED ELECTRONICALLY

The content of the electronically submitted sequence listing (Name: U.S. Plant Pat. No. 7,467,229_Replacement_SequenceListing_ST26.xml; Size: 26,137 bytes; and Date of Creation: Mar. 8, 2025) is herein incorporated by reference in its entirety.

### BACKGROUND

Obesity is a disease that negatively affects health due to abnormal or excessive fat accumulation and is one of the top 10 health risk factors designated by WHO. Obesity not only causes weight gain, but also causes diseases such as type 2 diabetes, dyslipidemia, high blood pressure, fatty liver, coronary artery disease, and colon cancer, and also causes psychological diseases such as psychological atrophy and depression.

There are three treatments for obesity approved by both the U.S. Food and Drug Administration (FDA) and the European Medicines Agency (EMA) in 2021. These are liraglutide, orlistat, and a combination of liraglutide and bupropion. Among these, liraglutide is known under the product name Saxenda, and was approved by the FDA in 2014, the EMA in 2015, and the Ministry of Food and Drug Safety (MFDS) of Korea in 2017. Since then, liraglutide has maintained its No. 1 position in the global obesity treatment market. Liraglutide is an analog of glucagon-like peptide-1 (GLP-1) that binds to GLP-1 receptors and sends signals. GLP-1, which is a hormone naturally secreted by the body in response to food intake, promotes insulin secretion from the pancreas to remove excess blood sugar and lowers glucagon secretion to regulate sugar secretion in the blood. It plays an important role in maintaining normal blood sugar levels and reduces and suppresses appetite by binding to receptors in the brain and transmitting signals to the hypothalamus. However, in addition to side effects such as diarrhea and vomiting, some cases of side effects have been reported in other tissues such as the liver, pancreas, and heart. Additionally, cases of increased incidence of some cancers, such as breast cancer and pancreatic cancer, have also been reported.

In contrast, insulin resistance syndrome is a cluster syndrome in which more than several clinical characteristics, such as decreased glucose metabolism, resistance to glucose utilization, hyperinsulinemia, impaired glucose tolerance, hyperglycemia, hypertension, increased triglycerides, and decreased HDL-cholesterol, exist in complex aspects. When insulin resistance persists, the insulin secretion capacity reaches its limit and insulin secretion decreases, making it impossible to inhibit the increase in blood sugar levels after a meal, thereby causing fasting blood sugar levels to rise above the normal range. When the amount of insulin secreted is not sufficient to overcome insulin resistance, a state of impaired glucose tolerance develops, whereas when the lack of insulin secretion is severe, it develops into diabetes with persistent high blood sugar levels.

The most important thing in discovering a substance that can treat or prevent a specific disease, such as obesity or insulin resistance syndrome, is to ensure a meaningful effect in reducing the severity of the disease. However, at the same time, there should be no side effects. Based on this, various research teams around the world have been conducting research to discover treatments for obesity or insulin resistance syndrome that have high safety and proven efficacy.

Flavonoids are secondary metabolites produced by plants and are contained in various fruits and vegetables. Flavonoids can be divided into two types: a glycoside form, in which sugar is attached, and an aglycone form, in which sugar is not attached. Most flavonoids that exist in nature take the form of glycosides with sugars attached. When flavonoids are ingested orally, glycosides are hydrolyzed to aglycone in the small or large intestine and then absorbed. However, since humans lack enzymes that can absorb or hydrolyze glycosides on their own, most glycosides are stored in the small intestine, they cannot be absorbed but are hydrolyzed into aglycone form by intestinal microorganisms such as Bacteroides spp, Lactobacillus spp., and Enterococcus spp. in the large intestine and then absorbed into enterocytes. When glycosides are maintained without hydrolysis, most of the flavonoids again escape out of the body.

Flavonoids are already known to have antioxidant, anti-inflammatory, and anticancer effects through various studies, but due to their low bioavailability, there are limitations in using them as candidate substances for treatment of diseases. One of the ways to solve the low bioavailability of flavonoids is to use microorganisms that can hydrolyze glycoside flavonoids into aglycones. The enzymes, which play the most central role in hydrolyzing rutin (i.e., a glycoside of quercetin) to form the aglycone form of quercetin, are-L-rhamnosidase (EC 3.2.1.40) and a-D-glucosidase (EC 3.2.1.21). In fact, there are study results showing the hydrolysis of rutin into quercetin in vitro using various microorganisms, including specific lactic acid bacteria.

The present inventors have discovered that when the Lactobacillus plantarum HAC03 strain and rutin (i.e., a type of flavonoid) are administered in combination, the anti-obesity effect and the effect of improving insulin resistance are significantly excellent due to a synergistic effect between the Lactobacillus plantarum HAC03 strain and rutin, thereby completing the present disclosure.

### SUMMARY

### Technical Problems

The problem to be solved in the present disclosure is to provide a pharmaceutical composition or health functional food composition, which has the effects of preventing or treating obesity or insulin resistance syndrome containing the Lactobacillus plantarum HAC03 strain and rutin as active ingredients.

### Technical Solution

In order to solve the above problems, the present disclosure provides a pharmaceutical composition for preventing or treating obesity or insulin resistance syndrome containing the Lactobacillus plantarum HAC03 strain (Accession Number: KCTC 13242BP) and rutin as active ingredients.

The composition can reduce one or more selected from the group consisting of total cholesterol, triglycerides, low density lipoprotein cholesterol (LDL), alanine aminotransferase (ALT), and aspartate aminotransferase (AST).

The composition can exhibit an anti-obesity effect by regulating the expression levels of one or more genes selected from the group consisting of fatty acid synthase (FAS), acetyl CoA carboxylase (ACC), peroxisome proliferator-activated receptor γ (PPARγ), sterol regulatory element binding protein 1C (SREBP1C), peroxisome proliferator-activated receptor α (PPARα), peroxisome proliferator-activated receptor γ coactivator 1 α(PGC1α), carnitine palmitoyltransferase 1 (CPT1), acyl-CoA oxidase 1 (ACOX1), uncoupling protein 1 (UCP1), and PR/SET domain 16 (PRDM16).

The anti-obesity effect may be one or more selected from the group consisting of inhibition of lipogenesis, increase in fat oxidation, and increase in body thermogenesis.

The composition may reduce blood sugar fluctuations, and reduce fasting blood sugar levels and fasting insulin levels.

The composition may exhibit the effect of reducing insulin resistance by controlling the expression level of glucose-6-phosphate dehydrogenase (G6P) gene or phosphoenolpyruvate carboxykinase (PEPCK) gene.

Additionally, the present disclosure provides a health functional food composition for preventing or improving obesity or insulin resistance syndrome, containing the Lactobacillus plantarum HAC03 strain (Accession number: KCTC 13242BP) and rutin as active ingredients.

The composition may reduce one or more selected from the group consisting of total cholesterol, triglycerides, low density lipoprotein cholesterol (LDL), alanine aminotransferase (ALT), and aspartate aminotransferase (AST).

The composition may exhibit an anti-obesity effect by regulating the expression levels of one or more genes selected from the group consisting of fatty acid synthase (FAS), acetyl CoA carboxylase (ACC), peroxisome proliferator-activated receptor γ (PPARγ), sterol regulatory element binding protein 1C (SREBP1C), peroxisome proliferator-activated receptor α (PPARα), peroxisome proliferator-activated receptor γ coactivator 1 α(PGC1α), carnitine palmitoyltransferase 1 (CPT1), acyl-CoA oxidase 1 (ACOX1), uncoupling protein 1 (UCP1), and PR/SET domain 16 (PRDM16).

The anti-obesity effect may be at least one effect selected from the group consisting of inhibition of lipogenesis, increase in fat oxidation, and increase in body thermogenesis.

The composition may reduce blood sugar fluctuations, and reduce fasting blood sugar levels and fasting insulin levels.

The composition may exhibit the effect of reducing insulin resistance by controlling the expression level of glucose-6-phosphate dehydrogenase (G6P) gene or phosphoenolpyruvate carboxykinase (PEPCK) gene.

### EFFECT OF INVENTION

The combination of the Lactobacillus plantarum HAC03 strain and rutin of the present disclosure has the efficacies of reducing body weight gain and tissue weight, reducing tissue cell size, inhibiting lipogenesis, and increasing fat oxidation. Additionally, the pharmaceutical composition has the efficacies of reducing blood sugar fluctuations and reducing fasting blood sugar levels and fasting insulin levels, and thus it can be used as a therapeutic agent for obesity or insulin resistance syndrome, a health functional food, etc.

### BRIEF DESCRIPTION OF THE DRAWINGS

FIG. 1 shows a schematic diagram of an animal experiment method.

FIG. 2 shows a graph confirming the change in body weight gain when the Lactobacillus plantarum HAC03 strain and rutin were administered in combination.

FIG. 3 shows graphs confirming the change in tissue weight when the Lactobacillus plantarum HAC03 strain and rutin were administered in combination, in which FIG. 3A shows the weight change in the liver, FIG. 3B shows the weight change in subcutaneous adipose tissue (SAT), FIG. 3C shows the weight change in epididymal adipose tissue (EAT), FIG. 3D shows the weight change in mesenteric adipose tissue (MAT), and FIG. 3E shows the weight change in brown adipose tissue (BAT).

FIG. 4 shows graphs confirming the change in tissue cell size when the Lactobacillus plantarum HAC03 strain and rutin were administered in combination, in which FIG. 4A shows the cell size change in the liver, FIG. 4B shows the cell size change in subcutaneous adipose tissue (SAT), FIG. 4C shows the cell size change in epididymal adipose tissue (EAT), FIG. 4D shows the cell size change in mesenteric adipose tissue (MAT), and FIG. 4E shows the cell size change in brown adipose tissue (BAT).

FIG. 5 shows graphs confirming the change in obesity-related biochemical indicators when the Lactobacillus plantarum HAC03 strain and rutin were administered in combination, in which FIG. 5A shows the change in total cholesterol, FIG. 5B shows the change in triglycerides, and FIG. 5C shows the change in low density lipoprotein cholesterol (LDL).

FIG. 6 shows graphs confirming the change in cytokines involved in lipogenesis in the extracted fat tissue when the Lactobacillus plantarum HAC03 strain and rutin were administered in combination, in which FIG. 6A shows the expression level change in fatty acid synthase (FAS), FIG. 6B shows the expression level change in acetyl CoA carboxylase (ACC), FIG. 6C shows the expression level change in peroxisome proliferator-activated receptor γ (PPARγ), and FIG. 6D shows the expression level change in sterol regulatory element binding protein 1C (SREBP1C).

FIG. 7 shows graphs confirming the change in cytokines involved in β-oxidation in the extracted fat tissue when the Lactobacillus plantarum HAC03 strain and rutin were administered in combination, in which FIG. 7A shows the expression level change in peroxisome proliferator-activated receptor γ (PPARγ), FIG. 7B shows the expression level change in peroxisome proliferator-activated receptor γ coactivator 1 α(PGC1α), FIG. 7C shows the expression level change in carnitine palmitoyltransferase 1 (CPT1), and FIG. 7D shows the expression level change in acyl-CoA oxidase 1 (ACOX1).

FIG. 8 shows graphs confirming the change in cytokines involved in thermogenesis in brown adipose tissue (BAT) when the Lactobacillus plantarum HAC03 strain and rutin were administered in combination, in which FIG. 8A shows the expression level change in uncoupling protein 1 (UCP1) and FIG. 8B shows the expression level change in PR/SET domain 16 (PRDM16).

FIG. 9 shows graphs confirming the change in blood sugar levels in an oral glucose tolerance test when the Lactobacillus plantarum HAC03 strain and rutin were administered in combination, in which FIG. 9A shows the blood sugar levels and FIG. 9B shows the values in area under curve (AUC).

FIG. 10 shows graphs confirming the change in fasting blood sugar levels and fasting insulin levels when the Lactobacillus plantarum HAC03 strain and rutin were administered in combination, in which FIG. 10A shows the blood sugar levels, FIG. 10B shows the change in insulin levels, and FIG. 10C shows the HOMA-IR levels.

FIG. 11 shows graphs confirming the change in the liver health-related biochemical indicators when the Lactobacillus plantarum HAC03 strain and rutin were administered in combination, in which FIG. 11A shows the change in alanine aminotransferase (ALT) and FIG. 11B shows the change in aspartate aminotransferase (AST).

FIG. 12 shows graphs confirming the change in cytokines involved in insulin resistance in the extracted adipose tissue when the Lactobacillus plantarum HAC03 strain and rutin were administered in combination, in which FIG. 12A shows the change in the expression level of glucose-6-phosphate dehydrogenase (G6P) and FIG. 12B shows the change in the expression level of phosphoenolpyruvate carboxykinase (PEPCK).

FIGS. 13A, 13B, 13C and 14 show the results of measuring intestinal microbial diversity, in which FIG. 13A, 13B, 13C show the results of alpha diversity recovery and FIG. 14 shows the results of beta diversity recovery.

FIGS. 15A and 15B show the results of observation of the relative distribution of intestinal microorganisms at the Phylum (A) or Class (B) level.

FIG. 16 shows the analysis of the relative distribution of intestinal microorganisms, in which FIG. 16A shows the relative distribution of Oscillospiraceae, FIG. 16B shows the relative distribution of Lachnospiraceae, and FIG. 16C shows the relative distribution of Eubacterium Coprostanoligenes.

### DETAILED DESCRIPTIONS OF EXEMPLARY EMBODIMENTS

The present inventors have confirmed that when the Lactobacillus plantarum HAC03 strain and rutin were administered in combination, the anti-obesity effect and the effect of improving insulin resistance were remarkably excellent due to the synergistic effect of the Lactobacillus plantarum HAC03 strain and rutin, thereby completing the present disclosure. Specifically, it was experimentally confirmed that when the above strain and rutin were administered in combination, a remarkably excellent anti-obesity effect was exhibited compared to when the strain or rutin was administered alone, due to a synergistic effect of the anti-obesity effect (which is possessed by the strain itself) and the anti-obesity effect (which is exhibited by rutin), and that the effect of improving insulin resistance was also remarkably excellent.

Therefore, in one aspect, the present disclosure relates to a pharmaceutical composition for preventing or treating obesity or insulin resistance syndrome, which contains the Lactobacillus plantarum HAC03 strain (Accession Number: KCTC 13242BP) and rutin as active ingredients.

Lactobacillus plantarum of the present disclosure may be used synonymously with Lactoplantibacillus plantarum.

The Lactobacillus plantarum HAC03 (Accession Number: KCTC 13242BP) strain was isolated from white kimchi and was confirmed to have excellent acid resistance and bile resistance, no antibiotic resistance, and no ability to produce biogenic amines (KR Registered Patent No. 10-2266314). Additionally, the Lactobacillus plantarum HAC03 strain is characterized in that it includes a 16s rDNA sequence represented by SEQ ID NO: 1 below.

TABLE 1
(SEQ ID NO: 1)
CTCTGGTATTGATTGGTGCTTGCATCATGATTTACATTTGAGTGAGTGGC
GAACTGGTGAGTAACACGTGGGAAACCTGCCCAGAAGCGGGGGATAACAC
CTGGAAACAGATGCTAATACCGCATAACAACTTGGACCGCATGGTCCGAG
CTTGAAAGATGGCTTCGGCTATCACTTTTGGATGGTCCCGCGGCGTATTA
GCTAGATGGTGGGGTAACGGCTCACCATGGCAATGATACGTAGCCGACCT
GAGAGGGTAATCGGCCACATTGGGACTGAGACACGGCCCAAACTCCTACG
GGAGGCAGCAGTAGGGAATCTTCCACAATGGACGAAAGTCTGATGGAGCA
ACGCCGCGTGAGTGAAGAAGGGTTTCGGCTCGTAAAACTCTGTTGTTAAA
GAAGAACATATCTGAGAGTAACTGTTCAGGTATTGACGGTATTTAACCAG
AAAGCCACGGCTAACTACGTGCCAGCAGCCGCGGTAATACGTAGGTGGCA
AGCGTTGTCCGGATTTATTGGGCGTAAAGCGAGCGCAGGCGGTTTTTTAA
GTCTGATGTGAAAGCCTTCGGCTCAACCGAAGAAGTGCATCGGAAACTGG
GAAACTTGAGTGCAGAAGAGGACAGTGGAACTCCATGTGTAGCGGTGAAA
TGCGTAGATATATGGAAGAACACCAGTGGCGAAGGCGGCTGTCTGGTCTG
TAACTGACGCTGAGGCTCGAAAGTATGGGTAGCAAACAGGATTAGATACC
CTGGTAGTCCATACCGTAAACGATGAATGCTAAGTGTTGGAGGGTTTCCG
CCCTTCAGTGCTGCAGCTAACGCATTAAGCATTCCGCCTGGGGAGTACGG
CCGCAAGGCTGAAACTCAAAGGAATTGACGGGGGCCCGCACAAGCGGTGG
AGCATGTGGTTTAATTCGAAGCTACGCGAAGAACCTTACCAGGTCTTGAC
ATACTATGCAAATCTAAGAGATTAGACGTTCCCTTCGGGGACATGGATAC
AGGTGGTGCATGGTTGTCGTCAGCTCGTGTCGTGAGATGTTGGGTTAAGT
CCCGCAACGAGCGCAACCCTTATTATCAGTTGCCAGCATTAAGTTGGGCA
CTCTGGTGAGACTGCCGGTGACAAACCGGAGGAAGGTGGGGATGACGTCA
AATCATCATGCCCCTTATGACCTGGGCTACACACGTGCTACAATGGATGG
TACAACGAGTTGCGAACTCGCGAGAGTAAGCTAATCTCTTAAAGCCATTC
TCAGTTCGGATTGTAGGCTGCAACTCGCCTACATGAAGTCGGAATCGCTA
GTAATCGCGGATCAGCATGCCGCGGTGAATACGTTCCCGGGCCTTGTACA
CACCGTCCGTCACAACCATGAGAGTTTGTAACACCCAAAGTCGGTGGGGT
AACC

Additionally, the Lactobacillus plantarum HAC03 strain possesses a gene that expresses an enzyme that plays a central role in hydrolyzing rutin. The rutin is a glycoside in which rutinose (a disaccharide made of glucose and rhamnose) is bound to carbon number 3 of quercetin. It was discovered in plants of the Ruta genus of the Rutaceae family, and was later isolated from many types of plants, including the flower buds of Sophora japonica in the legume family, buckwheat (Fagopyrum esculentum) in the Polygonaceae family, etc.

Through embodiments of the present invention, it was confirmed that the combined administration of the Lactobacillus plantarum HAC03 strain and rutin showed an anti-obesity effect (Example 1) and an effect of improving insulin resistance (Example 2), and it was confirmed that the combined administration was superior to the cases where the strain or rutin was administered alone. This shows that a composition containing the Lactobacillus plantarum HAC03 strain and rutin as active ingredients can prevent or treat obesity or insulin resistance syndrome.

Specifically, when the Lactobacillus plantarum HAC03 strain and rutin were administered in combination, there were reductions in body weight gain, tissue weight, and tissue cell size. Additionally, there were also reductions in total cholesterol, triglycerides, and low density lipoprotein cholesterol (LDL), which are obesity-related biochemical indicators. Moreover, the expression levels of lipogenesis-related genes (i.e., FAS, ACC, PPARγ, and SREBP1C) increased, whereas the expression levels of fat oxidation-related genes (i.e., PPARα, PGC1α, CPT1, and ACOX1) increased, and the expression level of genes involved in thermogenesis in brown adipose tissue (i.e., UCP1 and PRDM16) increased. (Example 1).

Additionally, when the Lactobacillus plantarum HAC03 strain and rutin were administered in combination, the changes in blood sugar levels were reduced, fasting blood sugar levels and insulin levels were reduced, and alanine aminotransferase (ALT) and aspartate aminotransferase (AST) (i.e., liver health-related biochemical indicators) were reduced. Additionally, the expression levels of G6P and PEPCK, which are gluconeogenesis-related genes, increased (Example 2).

Additionally, when the Lactobacillus plantarum HAC03 strain and rutin were administered in combination, the diversity and evenness of microbial species increased, and Eubacterium Coprostanoligenes, which significantly increased in obese patients, showed opposite trends to the microbial groups Oscillospiraceae and Lachnospiraceae, which are significantly decreased in obese patients (Example 3).

As used herein, the term “prevention” refers to any action that inhibits a disease or delays its onset by administering a pharmaceutical composition.

As used herein, the term “treatment” refers to abrogating, substantially inhibiting, slowing, or reversing the progression of a disease; substantially improving the clinical or aesthetic symptoms of the disease; or substantially preventing the appearance of clinical or aesthetic symptoms of the disease.

The pharmaceutical composition may have any one formulation selected from the group consisting of tablets, pills, powders, granules, capsules, suspensions, oral solutions, emulsions, syrups, sterilized aqueous solutions, non-aqueous solvents, suspensions, freeze-dried agents, and suppositories, and it may be in various oral or parenteral dosage forms. When formulated, the pharmaceutical composition is prepared using diluents or excipients, such as commonly used fillers, extenders, binders, wetting agents, disintegrants, and surfactants.

As used herein, the term “administration” refers to introducing the pharmaceutical composition of the present disclosure to a subject by any appropriate method, and the administration route may be oral or parenteral, as long as it can reach the target tissue.

Solid preparations for oral administration include tablets, pills, powders, granules, capsules, etc. These solid preparations may be prepared by mixing at least one excipient, such as starch, calcium carbonate, sucrose or lactose, and gelatin. Additionally, in addition to simple excipients, lubricants, such as magnesium stearate, and talc, may also be used. Liquid preparations for oral administration may include suspensions, oral solutions, emulsions, and syrups, and various excipients, such as wetting agents, sweeteners, fragrances, and preservatives, may be included in addition to the commonly used simple diluents such as water and liquid paraffin. Preparations for parenteral administration include sterilized aqueous solutions, non-aqueous solvents, suspensions, emulsions, freeze-dried preparations, and suppositories. Non-aqueous solvents and suspensions may include propylene glycol, polyethylene glycol, vegetable oil (e.g., olive oil), and injectable ester (e.g., ethyl oleate). As a base for suppositories, witepsol, macrogol, tween 61, cacao butter, laurine butter, glycerogelatin, etc. may be used.

The pharmaceutical composition of the present disclosure may be administered in a pharmaceutically effective amount. As used herein, the term “pharmaceutically effective amount” refers to an amount sufficient to treat a disease with a reasonable benefit/risk ratio applicable to medical treatment, and the effective dose level may be determined by factors including the type of individual, severity, age, sex, type of disease, activity of drug, sensitivity to drug, time of administration, route of administration and excretion rate, duration of treatment, drugs used simultaneously, and other factors well known in the medical field. The composition of the present disclosure may be administered as an individual therapeutic agent or in combination with other therapeutic agents, and may be administered sequentially or simultaneously with conventional therapeutic agents. Additionally, it may be administered single or multiple times. Considering all of the above factors, it is important to administer an amount that can achieve the maximum effect with the minimum amount without side effects, and this may be easily determined by a person skilled in the art.

The pharmaceutical composition of the present disclosure is not particularly limited as long as it is applied to any subject intended for the treatment of obesity or insulin resistance syndrome. For example, it may be used in any non-human animals (e.g., monkeys, dogs, cats, rabbits, guinea pigs, rats, mice, cows, sheep, pigs, goats, etc.), humans, birds, fish, etc. The pharmaceutical composition may be administered parenterally, subcutaneously, intraperitoneally, intrapulmonarily, and intranasally, and for local treatment, if necessary, may be administered by any suitable method, including intralesional administration. The preferred dose of the pharmaceutical composition of the present disclosure varies depending on the condition and weight of the individual, degree of disease, form of drug, route and period of administration, but may be appropriately selected by a person skilled in the art. For example, the pharmaceutical composition may be administered orally, rectally, or by intravenous, intramuscular, subcutaneous, intrauterine dura mater, or intracerebroventricular injection, but the administration method is not limited thereto.

The appropriate total daily dose may be determined by treatment within the range of sound medical judgment, and is generally in an amount of 0.001-1000 mg/kg, preferably 0.05-200 mg/kg, or more preferably 0.1-100 mg/kg. The amount may be administered once or several times a day.

From another aspect, the present disclosure relates to a health functional food composition for preventing or improving obesity or insulin resistance syndrome, containing the Lactobacillus plantarum HAC03 strain (Accession Number: KCTC 13242BP) and rutin as active ingredients.

The food composition refers to a natural product or processed product containing one

or more nutrients, preferably one which is in a state to be eaten directly after a certain degree of processing, and in the usual sense, it refers to one which includes all of foods, food additives, health functional foods, and functional beverages.

The food composition of the present disclosure may be prepared in the form of pills, powders, granules, infusions, tablets, capsules, or liquids, and there is no particular limitation on the type of food, for example, it may be prepared in the form of various beverages, gums, teas, vitamin complexes, health supplementary foods, etc.

Other ingredients may be added to the food composition, and their types are not particularly limited. For example, as in conventional foods, the ingredients may contain various herbal extracts, sitologically acceptable food supplementary additives, natural carbohydrates, etc. as additional ingredients, but the ingredients are not limited thereto.

As used herein, the term “food supplementary additive” refers to a component that can be added to food as a supplementary agent, and may be appropriately selected and used by a person skilled in the art as it is added to prepare each type of health functional food. Examples of food supplementary additives include various nutrients, vitamins, minerals (electrolytes), flavoring agents such as synthetic and natural flavors, colorants and fillers, pectic acid and its salts, alginic acid and its salts, organic acids, and protective colloidal thickeners, pH adjusters, stabilizers, preservatives, glycerin, alcohol, carbonating agents used in carbonated beverages, etc., but the types of food supplementary additives of the present disclosure are not limited to the above examples.

Examples of the natural carbohydrates include monosaccharides (e.g., glucose, fructose, etc.); disaccharides (e.g., maltose and sucrose, etc.); and polysaccharides (e.g., dextrin, cyclodextrin, etc.); sugar alcohols (e.g., xylitol, sorbitol, erythritol, etc.); and in addition to those described above, flavoring agents such as natural flavoring agents (e.g., thaumatin, etc.) stevia extracts (e.g., rebaudioside A, glycyrrhizin, etc.), and synthetic flavoring agents (saccharin, aspartame, etc.) may be used advantageously.

The food composition of the present disclosure may include health functional foods. As used herein, the term “health functional food” refers to food prepared and processed in the form of tablets, capsules, powders, granules, liquids, and pills using raw materials or ingredients with functional properties useful to the human body. In particular, functionality means controlling nutrients for the structure and function of the human body or obtaining useful effects for health purposes such as physiological effects. The health functional food of the present disclosure may be prepared by a method commonly used in the art, and may be prepared by adding raw materials and ingredients commonly added in the art. Additionally, unlike conventional drugs, the health functional food is prepared from food. Therefore, it has the advantage of having no side effects that may occur when taking a drug for a long time, and it may be highly portable.

Generally, the amount of the Lactobacillus plantarum HAC03 strain or rutin contained in the food composition may be added in the range of 0.1-90 wt % of the total weight of the food. However, in the case of long-term intake for the purpose of health and hygiene or health control, the amount may be below the above range. Additionally, it is possible that the food composition according to the present disclosure may contain, in addition to the Lactobacillus plantarum HAC03 strain or rutin, other ingredients that may preferably have a synergistic effect on the main effect within the range that does not impair the main effect aimed at by the present disclosure.

Hereinafter, the constitution and effects of the present disclosure will be described in more detail through examples. These examples are only for illustrating the present disclosure, and the scope of the present disclosure is not limited by these examples.

### Experimental Methods

### 1. Isolation and Identification of Strains

After purchasing white kimchi that was undergoing fermentation about 10-15 days after preparation, the pH was measured to confirm the decrease in pH due to lactic acid production, and then the lactic acid bacteria were isolated. 90 mL of physiological saline (0.85% NaCl/L) was added to an autoclaved plastic bag, and 10 g of the white kimchi sample was added and mixed uniformly at 200 rpm for 5 minutes (Stomacher® 400 Circulator, Seward, UK). 1 mL was dispensed, serially diluted 10 times in 9 mL of physiological saline (0.85% NaCl/L), and spread on an MRS solid medium (a 5.5% Lactobacillus MRS broth, BD Difco, USA and 1.5% bacteriological agar, Affymetrix, USA). Thereafter, the resultant was grown in an incubator at 37° C. for 24-48 hours, isolated, and identified.

Then, while culturing the grown bacteria at 37° C. for 24-48 hours using 3% hydrogen peroxide, catalase activity and Gram staining analysis were performed. The taxonomic species of the catalase-negative strain was identified through bidirectional sequence analysis of the 16S rDNA gene. The identification of the nucleotide sequence of the strain was performed by requesting to Solgent Co., Ltd. (Korea).

As a result, the strain of the present disclosure isolated from white kimchi was identified as a catalase-negative, Gram-positive, rod-shaped Lactobacillus plantarum strain, and was named Lactobacillus plantarum HAC03. The strain was deposited at the Korea

Research Institute of Bioscience and Biotechnology on Dec. 28, 2020 (Accession Number: KCTC 13242BP).

### Animal Experiments

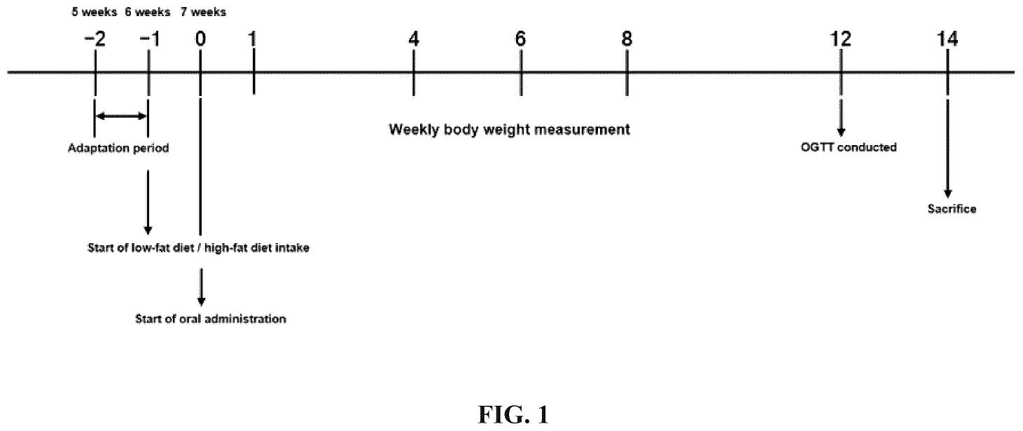

An animal experiment was performed to confirm the effect of the Lactobacillus plantarum HAC03 strain and rutin in preventing and improving obesity or insulin resistance syndrome. FIG. 1 shows a schematic diagram illustrating the method of animal experiments.

Five-week-old male C57BL6 mice were purchased and acclimated for one week. Then, the mice in each group (n=10) were fed a low-fat diet or high-fat diet. After first allowing them to consume a low-fat diet or high-fat diet for a week, the mice in each group were orally administered with Lactobacillus plantarum HAC03, quercetin, or rutin once a day for 14 weeks. The test groups were set as shown in Table 2 below.

TABLE 2

| | Number of | Dietary and Orally |
|---|---|---|
| Group | Individuals | Administered Materials |
| LFD | 10 | Low-fat diet (10% fat) |
| HFD | 10 | High-fat diet (60% fat) |
| Q | 10 | High-fat diet + Quercetin |
| | | (100 mg/kg) (Positive control) |
| R | 10 | High-fat diet + Rutin |
| | | (100 mg/kg) |
| H | 10 | High-fat diet + Lactobacillus plantarum |
| | | HAC03 Strain (3 × 10⁸ CFU/mouse) |
| HR | 10 | High-fat diet + Lactobacillus plantarum |
| | | HAC03 Strain (3 × 10⁸ CFU/mouse) + |
| | | Rutin (100 mg/kg) |
| HQ | 10 | High-fat diet + Lactobacillus plantarum |
| | | HAC03 Strain (3 × 10⁸ CFU/mouse) + |
| | | Quercetin (100 mg/kg) |

| | |
|---|---|
| Total | 70 |

Body weight and the amount of food consumed were measured for each group every week. After oral administration, glucose was administered orally at the 12th week and an oral glucose tolerance test was performed. After oral administration, at the 14th week, the animals were euthanized with carbon dioxide gas and their body weight and tissue weight were measured. Additionally, after extracting blood from the heart, serum was separated therefrom, and biochemical indicators related to obesity and liver health were measured using the serum. In particular, total cholesterol, triglycerides, and low density lipoprotein cholesterol (LDL) were used as obesity-related biochemical indicators, and alanine aminotransferase (ALT) and aspartate aminotransferase (AST) were used as liver health-related biochemical indicators. Additionally, fat tissue was extracted and subjected to H&E staining, and the size and number of fat cells were measured.

### 3. Measurement of Cytokines

In order to confirm the molecular mechanism involved in the effects of preventing and improving the Lactobacillus plantarum HAC03 strain and rutin on obesity or insulin resistance syndrome, RNA was identified and its cDNA was synthesized in adipose tissue. Then, the expression levels of lipogenesis-related genes were measured at the mRNA level through qPCR.

Specifically, the colons of mice in each experimental group were homogenized, dissolved in Trizol® (RNA extraction buffer, Thermo Fisher), and RNA was extracted according to the protocol provided by the manufacturer. The extracted RNA was quantified, and an equal amount of RNA was synthesized into cDNA by reverse transcription using the SuperiorScript III cDNA synthesis Kit (Enzynomics, Daejeon, South Korea). Thereafter, real-time PCR was performed using each gene-specific primer in Table 3, TOPreal qPCR 2X premix (SYBR Green with high ROX) (Enzynomics, Daejeon, South Korea), and cDNA. Real-time PCR was performed using the ABI 7500 Fast Real time PCR system (Applied Biosystems, Waltham, MA, USA). The relative gene expression levels were calculated using the ÄÄCt method.

TABLE 3

| | Forward | Reverse |
|---|---|---|
| Target | (5′→ 3′) | (5′→ 3′) |
| FAS | CTGGACTCGCTCATGGGTG | CATTTCCTGAAGTTTCCGCA |
| | (SEQ ID NO: 2) | G (SEQ ID NO: 3) |
| ACC | TGACAGACTGATCGCAGAGAA | TGGAGAGCCCCACACACA |
| | AG | (SEQ ID NO: 5) |
| | (SEQ ID NO: 4) | |
| PPARγ | AGTGGAGACCGCCCAGG | GCAGCAGGTTGTCTTGGATG |
| | (SEQ ID NO: 6) | T (SEQ ID NO: 7) |
| SREBP1C | AGCAGCCCCTAGAACAAACAC | CAGCAGTGAGTCTGCCTTGA |
| | (SEQ ID NO: 8) | T (SEQ ID NO: 9) |
| PPARα | GTACGGTGTGTATGAAGCCAT | GCCGTACGCGATCAGCAT |
| | CTT | (SEQ ID NO: 11) |
| | (SEQ ID NO: 10) | |
| PGC1 | CCTGAAGCCGGGAGAGAATG | TAGCCAGCAGAGACTGTGGA |
| | (SEQ ID NO: 12) | (SEQ ID NO: 13) |
| CPT1 | TGAGTGGCGTCCTCTTTGG | TCAGCGAGTAGCGCATAGTC |
| | (SEQ ID NO: 14) | A (SEQ ID NO: 15) |
| ACOX1 | GTGCAGCTCAGAGTCTGTCCA | TACTGCTGCGTCTGAAAATC |
| | A (SEQ ID NO: 16) | CA (SEQ ID NO: 17) |
| UCP1 | CTTTGCCTCACTCAGGATTGG | ACTGCCACACCTCCAGTCAT |
| | (SEQ ID NO: 18) | T (SEQ ID NO: 19) |
| PRDM16 | GAAGTCACAGGAGGACACGG | CTCGCTCCTCAACACACCTC |
| | (SEQ ID NO: 20) | (SEQ ID NO: 21) |
| G6P | CGACTCGCTATCTCCAAGTGA | GTTGAACCAGTCTCCGACCA |
| | (SEQ ID NO: 22) | (SEQ ID NO: 23) |
| PEPCK | CTGCATAACGGTCTGGACTTC | CAGCAACTGCCCGTACTCC |
| | (SEQ ID NO: 24) | (SEQ ID NO: 25) |
| Beta- | GGCACCACACYTTCTACAATG | GGGGTGTTGAAGGTCTCAAA |
| actin | (SEQ ID NO: 26) | C (SEQ ID NO: 27) |

### 4. Measurement of Insulin

Serum insulin levels were measured using the Ultra Sensitive Mouse Insulin ELISA kit (MORINAGA Institute of Biological Science, Inc. (MIoBS), Yokohama-shi, Japan).

### 5. Statistics

All data were expressed as mean±standard error of the median (SEM). Statistical significance was set as follows.

+p<0.05, ++p <0.01 between R and HR.

#p<0.05, ##p<0.01, ###p<0.001 between H and HR.

* p<0.05, ** p<0.01, *** p<0.001 compared with HFD.

### Example 1 Anti-Obesity Effect

### 1. Changes in Weight Gain

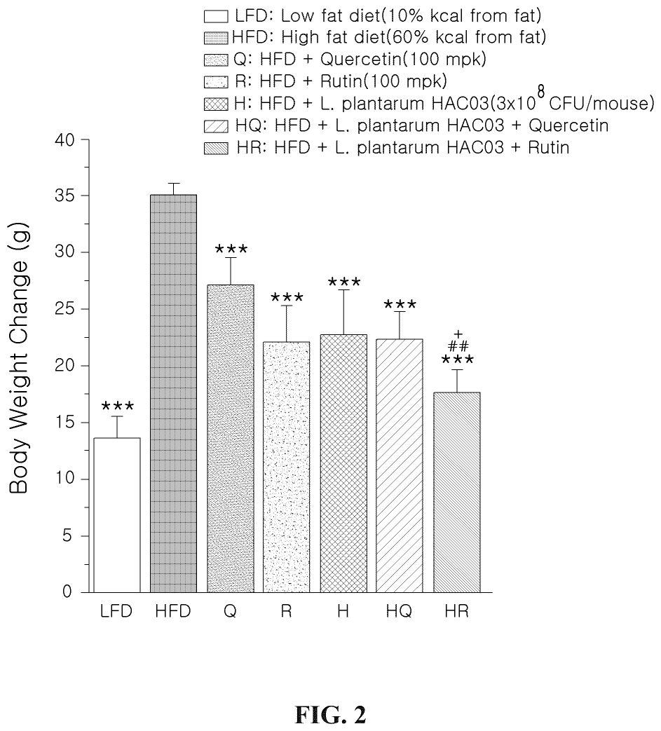

FIG. 2 confirms the change in body weight gain when the Lactobacillus plantarum HAC03 strain and rutin were administered in combination.

Specifically, it was confirmed that compared to the group, in which only a high-fat diet (HFD) was consumed, the change in body weight gain was reduced in all groups where the high-fat diet was administered in conjunction with the Lactobacillus plantarum HAC03 strain, rutin, or a combination of both. Additionally, in the group (HR), in which the Lactobacillus plantarum HAC03 strain and rutin (HR) were administered in combination, the change in body weight gain was significantly reduced compared to the groups where the Lactobacillus plantarum HAC03 strain (H) or rutin (R) was administered alone (FIG. 2).

### 2. Tissue Weight Changes

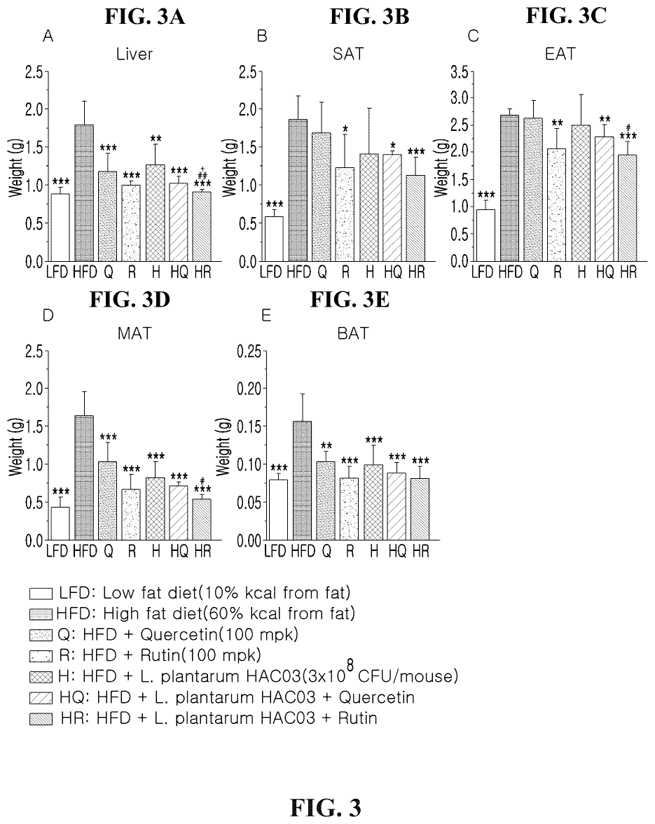

FIG. 3 confirms the change in tissue weight when the Lactobacillus plantarum HAC03 strain and rutin were administered in combination, in which FIG. 3A shows the weight change in the liver, FIG. 3B shows the weight change in subcutaneous adipose tissue (SAT), FIG. 3C shows the weight change in epididymal adipose tissue (EAT), FIG. 3D shows the weight change in mesenteric adipose tissue (MAT), and FIG. 3E shows the weight change in brown adipose tissue (BAT).

Specifically, it was confirmed that compared to the group, in which only a high-fat diet (HFD) was consumed, liver weight was reduced in all groups where a high-fat diet was administered in conjunction with the Lactobacillus plantarum HAC03 strain, rutin, or a combination of both. In the group (HR) where the Lactobacillus plantarum HAC03 strain and rutin (HR) were administered in combination, liver weight was significantly reduced compared to the groups where the Lactobacillus plantarum HAC03 strain (H) or rutin (R) was administered alone (FIG. 3A).

Additionally, it was confirmed that compared to the group, in which only a high-fat diet (HFD) was consumed, the weight of subcutaneous fat tissue was reduced in all groups where a high-fat diet was administered in conjunction with the Lactobacillus plantarum HAC03 strain, rutin, or a combination of both. In the group (HR) where the Lactobacillus plantarum HAC03 strain and rutin (HR) were administered in combination, the weight of subcutaneous fat tissue decreased compared to the groups where the Lactobacillus plantarum HAC03 strain (H) or rutin (R) was administered alone, but was not statistically significant (FIG. 3B).

Additionally, it was confirmed that compared to the group, in which only a high-fat diet (HFD) was consumed, the weight of epididymal adipose tissue (EAT) was reduced in all groups administered single or combined high-fat diet and Lactobacillus plantarum HAC03 strain or rutin. In the group (HR) where the Lactobacillus plantarum HAC03 strain and rutin were administered in combination, the weight of epididymal adipose tissue (EAT) was significantly reduced compared to the groups where the Lactobacillus plantarum HAC03 strain (H) was administered alone (FIG. 3C).

Additionally, it was confirmed that compared to the group, in which only a high-fat diet (HFD) was consumed, the weight of mesenteric adipose tissue was reduced in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the weight of mesenteric adipose tissue was significantly reduced compared to the group where the Lactobacillus plantarum HAC03 strain (H) was administered alone (FIG. 3D).

Additionally, it was confirmed that compared to the group, in which only a high-fat diet (HFD) was consumed, the weight of brown adipose tissue was reduced in all groups where a high-fat diet was administered in conjunction with the Lactobacillus plantarum HAC03 strain, rutin, or a combination of both. In the group (HR) where the Lactobacillus plantarum HAC03 strain and rutin (HR) were administered in combination, the weight of subcutaneous fat tissue was reduced compared to the groups where the Lactobacillus plantarum HAC03 strain (H) or rutin (R) was administered alone, but was not statistically significant (FIG. 3E).

### 3. Changes in Tissue Cell Size

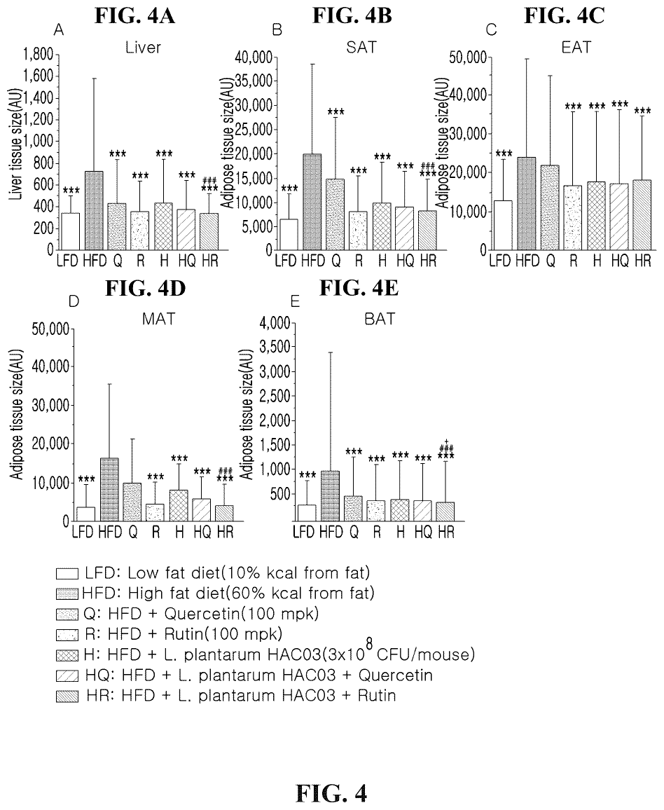

FIG. 4 confirms the change in tissue cell size when the Lactobacillus plantarum HAC03 strain and rutin were administered in combination, in which FIG. 4A shows the cell size change in the liver, FIG. 4B shows the cell size change in subcutaneous adipose tissue (SAT), FIG. 4C shows the cell size change in epididymal adipose tissue (EAT), FIG. 4D shows the cell size change in mesenteric adipose tissue (MAT), and FIG. 4E shows the cell size change in brown adipose tissue (BAT).

Specifically, it was confirmed that compared to the group, in which only a high-fat diet (HFD) was consumed, the liver tissue cell size was reduced in all groups where a high-fat diet was administered in conjunction with the Lactobacillus plantarum HAC03 strain, rutin, or a combination of both. In the group (HR) where the Lactobacillus plantarum HAC03 strain and rutin (HR) were administered in combination, the liver tissue cell size was significantly reduced compared to the group where the Lactobacillus plantarum HAC03 strain (H) was administered alone (FIG. 4A).

Additionally, it was confirmed that compared to the group, in which only a high-fat diet (HFD) was consumed, the size of subcutaneous fat tissue was reduced in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the cell size change in subcutaneous adipose tissue was significantly reduced compared to the group where the Lactobacillus plantarum HAC03 strain (H) was administered alone (FIG. 4B).

Additionally, it was confirmed that compared to the group, in which only a high-fat diet (HFD) was consumed, the size of epididymal adipose tissue was reduced in all groups where a high-fat diet was administered in conjunction with the Lactobacillus plantarum HAC03 strain, rutin, or a combination of both. In the group (HR) where the Lactobacillus plantarum HAC03 strain and rutin (HR) were administered in combination, the weight of epididymal adipose tissue was reduced compared to the groups where the Lactobacillus plantarum HAC03 strain (H) or rutin (R) was administered alone, but was not statistically significant (FIG. 4C).

Additionally, it was confirmed that compared to the group, in which only a high-fat diet (HFD) was consumed, the size of mesenteric adipose tissue was reduced in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the size of mesenteric adipose tissue was significantly reduced compared to the group where the Lactobacillus plantarum HAC03 strain (H) was administered alone (FIG. 4D).

Additionally, it was confirmed that compared to the group, in which only a high-fat diet (HFD) was consumed, the size of brown adipose tissue was reduced in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the size of brown adipose tissue was significantly reduced compared to the groups where the Lactobacillus plantarum HAC03 strain (H) or rutin (R) was administered alone (FIG. 4E).

### 4. Changes in Biochemical Indicators

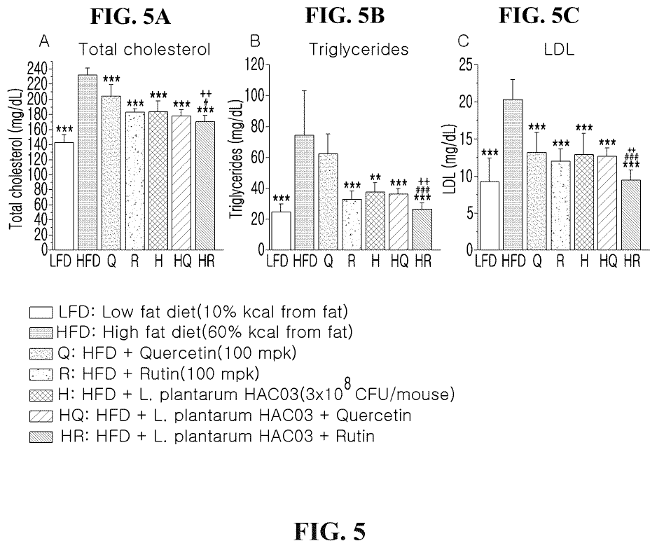

FIG. 5 confirms changes in obesity-related biochemical indicators when the Lactobacillus plantarum HAC03 strain and rutin were administered in combination, in which FIG. 5A shows the change in total cholesterol, FIG. 5B shows the change in triglycerides, and FIG. 5C shows the change in low density lipoprotein cholesterol (LDL).

Specifically, it was confirmed that compared to the group, in which only a high-fat diet (HFD) was consumed, the total cholesterol level was reduced in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the total cholesterol level was significantly reduced compared to the groups where the Lactobacillus plantarum HAC03 strain (H) or rutin (R) was administered alone.

Additionally, it was confirmed that compared to the group, in which only a high-fat diet (HFD) was consumed, the triglycerides level was reduced in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the triglycerides level was significantly reduced compared to the groups where the Lactobacillus plantarum HAC03 strain (H) or rutin (R) was administered alone.

Additionally, compared to the group, in which only a high-fat diet (HFD) was consumed, the low-density lipoprotein cholesterol (LDL) level was reduced in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the LDL level was significantly reduced compared to the groups where the Lactobacillus plantarum HAC03 strain (H) or rutin (R) was administered alone.

### 5. Changes in Gene Expression

### (1) Changes in Expression Level of Lipogenesis Genes

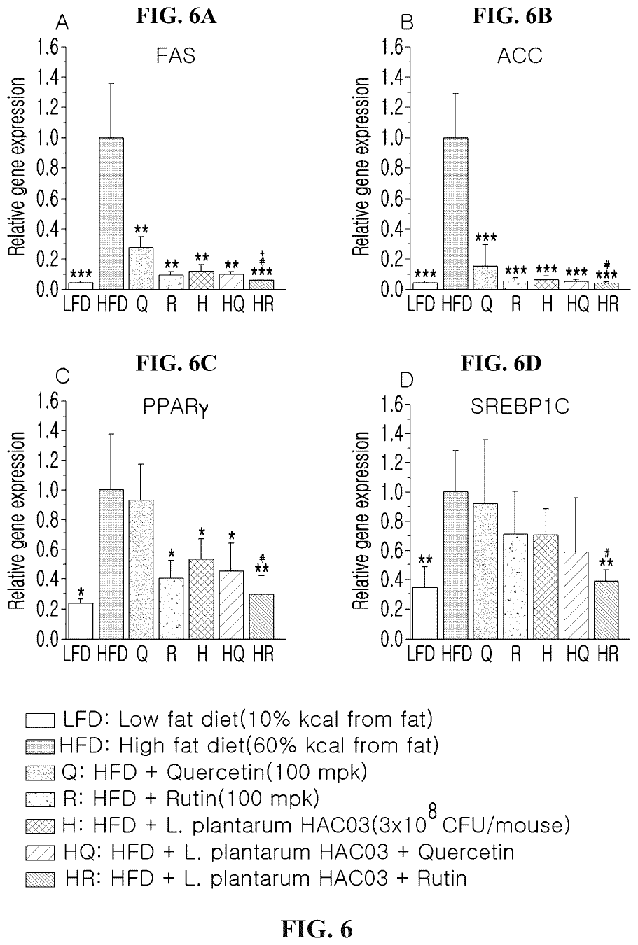

FIG. 6 shows changes in cytokines involved in lipogenesis in extracted fat tissue when the Lactobacillus plantarum HAC03 strain and rutin were administered in combination, in which FIG. 6A shows the expression level change in fatty acid synthase (FAS), FIG. 6B shows the expression level change in acetyl COA carboxylase (ACC), FIG. 6C shows the expression level change in peroxisome proliferator-activated receptor γ (PPARγ), and FIG. 6D shows the expression level change in sterol regulatory element binding protein 1C (SREBP1C). FAS, ACC, PPARγ, and SREBP1C are all genes involved in lipogenesis.

Specifically, it was confirmed that compared to the group, in which only a high-fat diet (HFD) was consumed, the FAS expression level was reduced in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the FAS expression level was significantly reduced compared to the group where the Lactobacillus plantarum HAC03 strain (H) was administered alone (FIG. 6A).

Additionally, it was confirmed that compared to the group, in which only a high-fat diet (HFD) was consumed, the ACC expression level was reduced in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the ACC expression level was significantly reduced compared to the group where the Lactobacillus plantarum HAC03 strain (H) was administered alone (FIG. 6B).

Additionally, it was confirmed that compared to the group, in which only a high-fat diet (HFD) was consumed, the expression level of PPARγ was significantly reduced in all groups where a high-fat diet was administered in conjunction with the Lactobacillus plantarum HAC03 strain, rutin, or a combination of both, except for the group (Q) where quercetin was administered alone. In the group (HR) where the Lactobacillus plantarum HAC03 strain and rutin were administered in combination, the expression level of PPARγ was significantly reduced compared to the group where the Lactobacillus plantarum HAC03 strain (H) was administered alone (FIG. 6C).

Additionally, compared to the group, in which only a high-fat diet (HFD) was consumed, a significant reduction in SREBP1C expression level was confirmed only in the group where a low-fat diet (LFD) was consumed and the group (HR) where the Lactobacillus plantarum HAC03 strain and rutin were administered in combination. In the group (HR) where the Lactobacillus plantarum HAC03 strain and rutin were administered in combination, the expression level of SREBP1C was significantly reduced compared to the group where the Lactobacillus plantarum HAC03 strain (H) was administered alone (FIG. 6D).

### (2) Changes in Expression Level Fat Oxidation-Related Genes

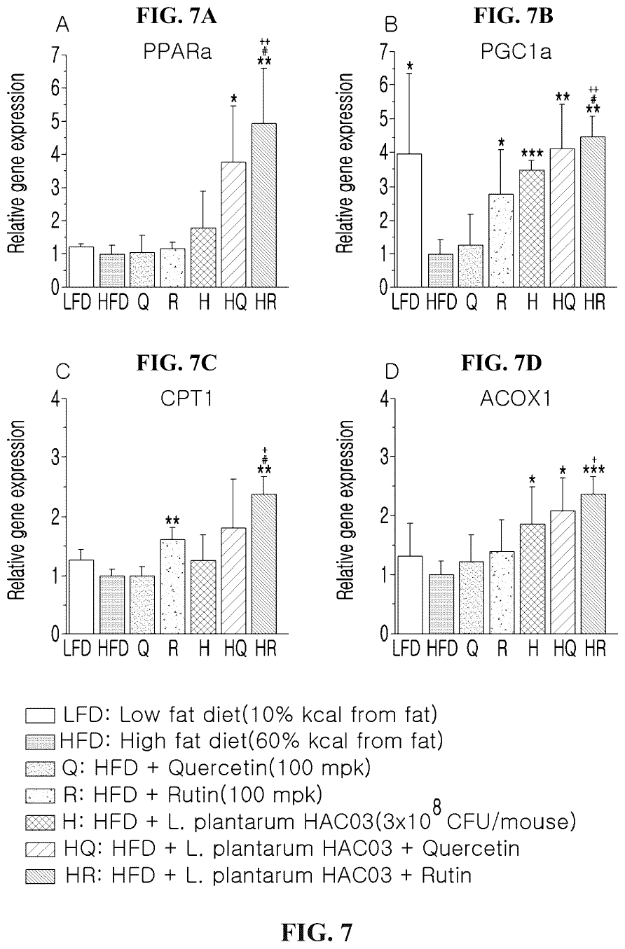

FIG. 7 confirms changes in cytokines involved in fat oxidation (β-oxidation) in extracted fat tissue when the Lactobacillus plantarum HAC03 strain and rutin were administered in combination, in which FIG. 7A shows the expression level change in peroxisome proliferator-activated receptor γ (PPARγ), FIG. 7B shows the expression level change in peroxisome proliferator-activated receptor γ coactivator 1 α (PGC1α), FIG. 7C shows the expression level change in carnitine palmitoyltransferase 1 (CPT1), and FIG. 7D shows the expression level change in acyl-CoA oxidase 1 (ACOX1). PPARα, PGC1α, CPT1, and ACOX1 are all genes involved in fat oxidation.

Specifically, compared to the group, in which only a high-fat diet (HFD) was consumed, the expression level of PPARα was significantly increased in the groups (HQ, HR) where the Lactobacillus plantarum HAC03 strain was administered in combination with quercetin or rutin. In the group (HR) where the Lactobacillus plantarum HAC03 strain and rutin were administered in combination, the expression level of PPARα was significantly increased compared to the groups (H, R) where the Lactobacillus plantarum HAC03 strain or rutin was administered alone (FIG. 7A).

Additionally, compared to the group, in which only a high-fat diet (HFD) was consumed, a significant increase in PGC1 expression was confirmed in all groups except the group (Q) where only quercetin was consumed. In the group (HR) where the Lactobacillus plantarum HAC03 strain and rutin were administered in combination, the expression level of PGC1 was significantly increased compared to the groups (H, R) where the Lactobacillus plantarum HAC03 strain or rutin was administered alone (FIG. 7B).

Additionally, it was confirmed that compared to the group, in which only a high-fat diet (HFD) was consumed, a significant increase in the expression level of CPT1 was confirmed in the group (R), where only rutin was administered, and the group (HR), where the Lactobacillus plantarum HAC03 strain and rutin were administered in combination. In the group (HR) where the Lactobacillus plantarum HAC03 strain and rutin were administered in combination, the expression level of CPT1 was significantly increased compared to the groups (H, R) where the Lactobacillus plantarum HAC03 strain or rutin was administered alone (FIG. 7C).

Additionally, compared to the group, in which only a high-fat diet (HFD) was consumed, a significant increase in the expression level of ACOX1 was confirmed in the group (H), where only the Lactobacillus plantarum HAC03 strain was administered, and the groups (HQ, HR), where the Lactobacillus plantarum HAC03 strain and rutin were administered in combination. In the group (HR) where the Lactobacillus plantarum HAC03 strain and rutin were administered in combination, the expression level of ACOX1 was significantly increased compared to the group (R), where rutin was administered alone (FIG. 7D).

### (3) Changes in Expression Level of Thermogenesis-Related Genes

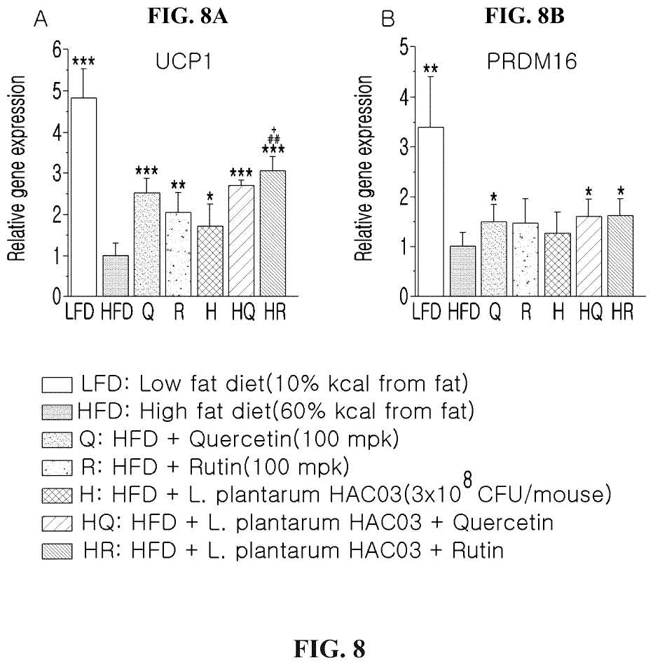

FIG. 8 confirms changes in cytokines involved in thermogenesis in brown adipose tissue (BAT) when Lactobacillus plantarum HAC03 strain and rutin are administered in combination, in which FIG. 8A shows the expression level change in uncoupling protein 1 (UCP1) and FIG. 8B shows the expression level change in PR/SET domain 16 (PRDM16). UCP1 and PRDM16 are involved in generating body heat and increasing energy use.

Specifically, it was confirmed that compared to the group, in which only a high-fat diet (HFD) was consumed, the expression level of UCP1 increased in brown adipose tissue (BAT) in all groups where a high-fat diet was administered in conjunction with the Lactobacillus plantarum HAC03 strain, rutin, or a combination of both. In the group (HR) where the Lactobacillus plantarum HAC03 strain and rutin were administered in combination, the expression level of UCP1 was significantly increased compared to the groups where the Lactobacillus plantarum HAC03 strain (H) or rutin (R) was administered alone (FIG. 8A).

Additionally, it was confirmed that compared to the group, in which only a high-fat diet (HFD) was consumed, the expression level of PRDM16 increased in brown adipose tissue (BAT), in the group where quercetin (Q) was consumed, and the group (HQ) where the Lactobacillus plantarum HAC03 strain and quercetin, were administered in combination. In the group (HR) where the Lactobacillus plantarum HAC03 strain and rutin were administered in combination, the expression level of PRDM16 increased even compared to the groups where the Lactobacillus plantarum HAC03 strain (H) or rutin (R) was administered alone, but was not statistically significant (FIG. 8B).

### Example 2 Effect of Improving Insulin Resistance

### 1. Changes in Blood Sugar and Insulin Levels

### (1) Confirmation of Reduction Of Change In Blood Sugar Levels

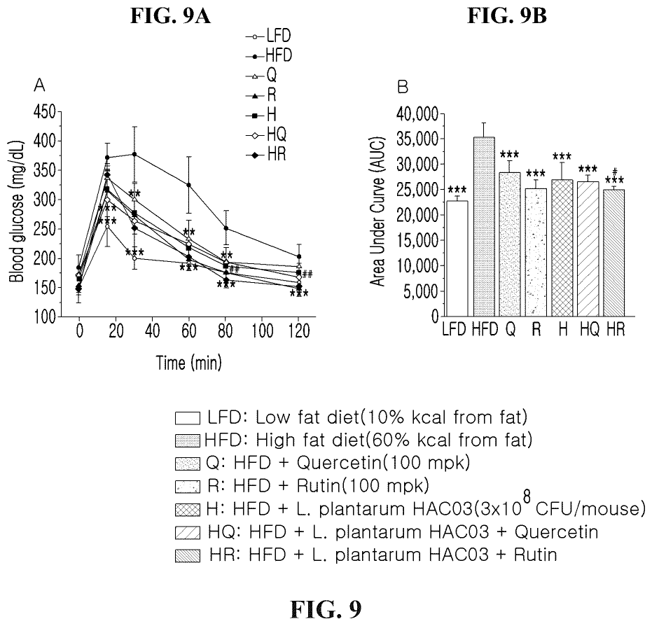

FIG. 9 shows changes in blood sugar levels as a result of an oral glucose tolerance test when the Lactobacillus plantarum HAC03 strain and rutin were administered in combination, in which FIG. 9A shows the blood sugar levels and FIG. 9B shows the values in area under curve (AUC).

Specifically, compared to the group, in which only a high-fat diet (HFD) was consumed, in all groups where a high-fat diet was administered in conjunction with the Lactobacillus plantarum HAC03 strain, rutin, or a combination of both, after glucose administration, the maximum blood sugar levels (FIG. 9A) and the AUC values (FIG. 9B) showed a statistically significant decrease. The group (HR), in which the Lactobacillus plantarum HAC03 strain and rutin were administered in combination, showed a statistically significant decrease in the AUC values even compared to the group where the Lactobacillus plantarum HAC03 strain (H) was administered alone.

### (2) Reduction in Fasting Blood Sugar and Insulin Levels

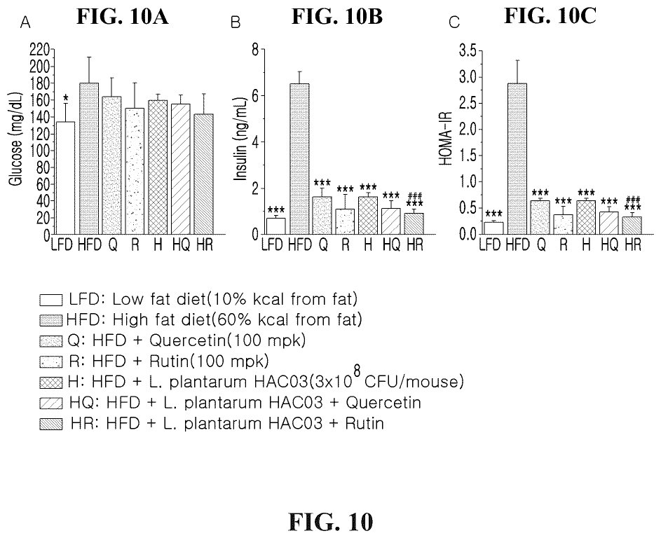

FIG. 10 shows changes in fasting blood sugar and insulin levels when the Lactobacillus plantarum HAC03 strain and rutin were administered in combination, in which FIG. 10A shows the blood sugar levels, FIG. 10B shows the change in insulin levels, and FIG. 10C shows the HOMA-IR levels.

Compared to the group, in which only a high-fat diet (HFD) was consumed, insulin levels and HOMA-IR levels, which indicate insulin resistance, decreased in all groups where a high-fat diet was administered in conjunction with the Lactobacillus plantarum HAC03 strain, rutin, or a combination of both. The group (HR), where the Lactobacillus plantarum HAC03 strain and rutin were administered in combination, showed a statistically significant decrease in the insulin and HOMA-IR levels compared to the group where the Lactobacillus plantarum HAC03 strain (H) was administered alone.

### 2. Changes in Biochemical Indicators

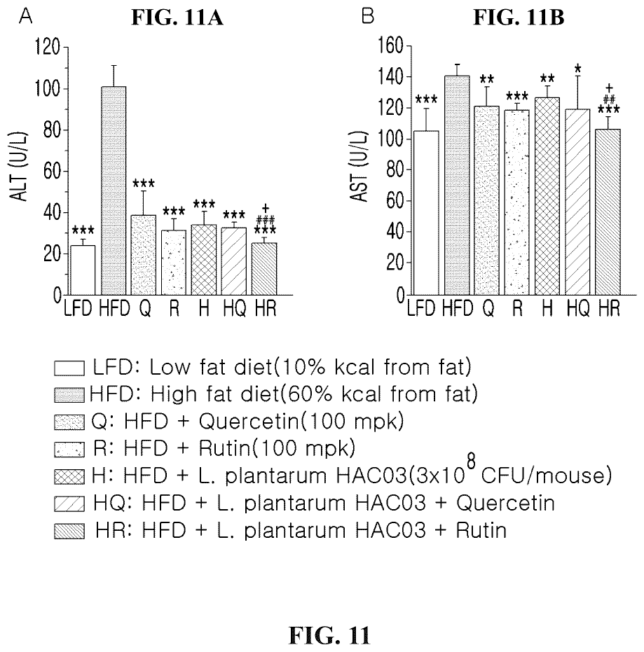

FIG. 11 confirms changes in liver health-related biochemical indicators in the cases where the Lactobacillus plantarum HAC03 strain and rutin were administered in combination, in which FIG. 11A shows the change in alanine aminotransferase (ALT) and FIG. 11B shows the change in aspartate aminotransferase (AST). Liver enzymes, ALT and AST, are important indicators of liver health because they leak into the blood when liver cells die.

Specifically, compared to the group, in which only a high-fat diet (HFD) was consumed, ALT and AST levels decreased in all groups where a high-fat diet was administered in conjunction with the Lactobacillus plantarum HAC03 strain, rutin, or a combination of both. In the group (HR) where the Lactobacillus plantarum HAC03 strain and rutin (HR) were administered in combination, significant decreases in ALT and AST levels were confirmed even compared to the groups where the Lactobacillus plantarum HAC03 strain (H) or rutin (R) was administered alone.

### 3. Changes in Gene Expression

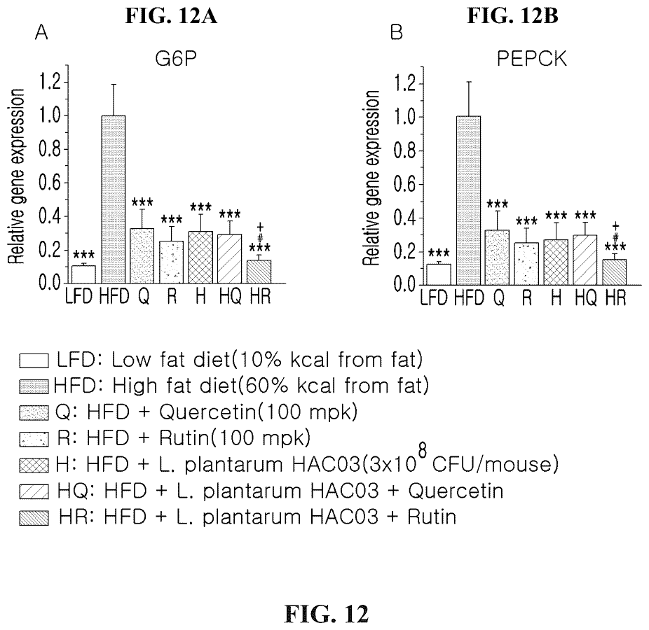

FIG. 12 shows changes in cytokines related to insulin resistance in extracted adipose tissue when the Lactobacillus plantarum HAC03 strain and rutin were administered in combination, in which FIG. 12A shows the change in the expression level of glucose-6-phosphate dehydrogenase (G6P) and FIG. 12B shows the change in the expression level of phosphoenolpyruvate carboxykinase (PEPCK). G6P is a gene expressing glucose-6-phosphate dehydrogenase, which is an enzyme that activates gluconeogenesis, and when G6P increases, blood sugar level increases. PEPCK is a gene expressing phosphoenolpyruvate carboxykinase, which is an enzyme that activates gluconeogenesis, and when PEPCK increases, blood sugar level increases.

Specifically, it was confirmed that compared to the group where only a high-fat diet (HFD) was consumed, the expression level of G6P decreased in all groups. In the group (HR) where the Lactobacillus plantarum HAC03 strain and rutin were administered in combination, the expression level of G6P significantly decreased even compared to the groups where Lactobacillus plantarum HAC03 strain (H) or rutin (R) was administered alone (FIG. 12A).

Additionally, it was confirmed that compared to the group where only a high-fat diet (HFD) was consumed, the expression level of PEPCK decreased in all groups. In the group (HR) where the Lactobacillus plantarum HAC03 strain and rutin were administered in combination, it was confirmed that the PEPCK expression level significantly decreased even compared to the groups where Lactobacillus plantarum HAC03 strain (H) or rutin (R) was administered alone (FIG. 12B).

### Example 3 Results of Improving Intestinal Microorganisms (Change in Diversity Recovery)

### Results of Diversity Measurement

### (1) Results of Alpha Diversity Recovery

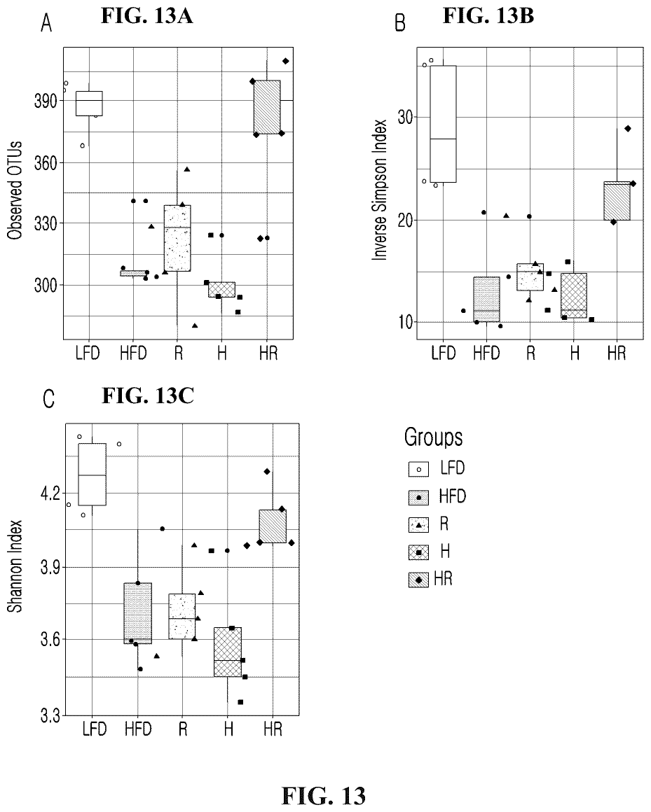

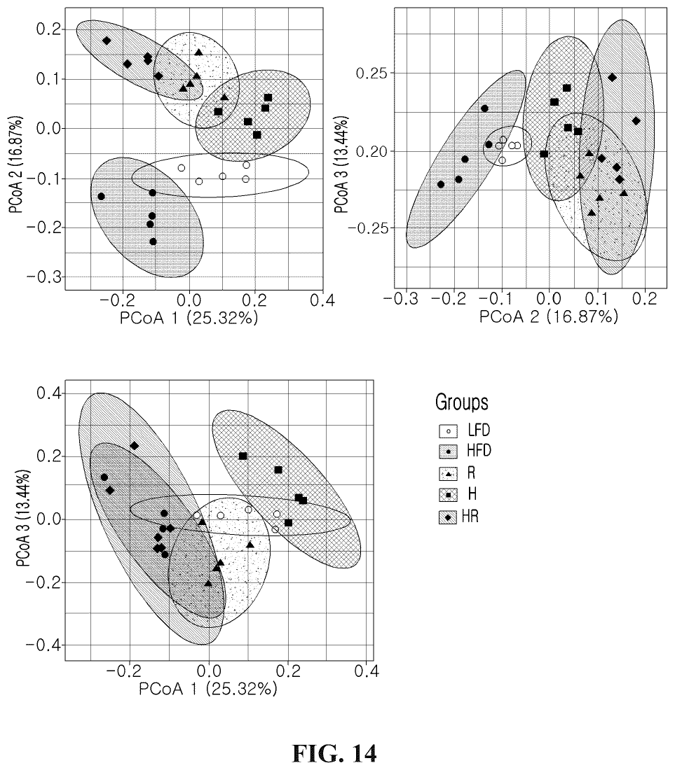

FIG. 13 shows the results of alpha diversity recovery, in which FIG. 13 shows the results of alpha diversity recovery and FIG. 14 shows the results of beta diversity recovery.

Observed OTUs is a measurement method that indicates the total number of operational taxonomic units (OTUs) or amplicon sequence variants (ASVs) present in a sample. The number of each OTU present was not considered. By observing the observed OTUs, it is possible to confirm that the type of intestinal microorganisms increased in the combination group (HR) of the Lactobacillus plantarum HAC03 strain and rutin, compared to the group where only a high-fat diet (HFD) was consumed (FIG. 13A).

The Simpson index is a measurement method that considers species diversity (richness) and evenness (evenness), and is a measurement method that places more weight on evenness.

Simpson index=0: means infinite diversity.

Simpson index=1: means no diversity.

Inverse Simpson index is simply an inverted value of the Simpson index and is set to make it easier to compare with other indices. By observing the inverse Simpson index, it is possible to confirm that the distribution of HR's intestinal microorganisms not only increased in type, but also increased evenly (FIG. 13B).

The Shannon index is a measurement method that considers the richness and evenness of species and is a measurement method that places more weight on diversity. The higher the value, the higher the species diversity.

Shannon index=0: means no diversity.

Shannon index=1: means infinite diversity.

By observing the Shannon index, it is possible to confirm that the distribution of HR's intestinal microorganisms not only increased in type, but also increased evenly (FIG. 13C).

Through the above, it was confirmed that species diversity and evenness increased in the group (HR) where the Lactobacillus plantarum HAC03 strain and rutin were administered in combination.

### (2) Results of Beta Diversity Recovery

FIG. 14 shows the results of beta diversity recovery. Beta diversity is an indicator of similarity between microbial groups. Based on the PCoA1 axis, it is possible to confirm that there was the smallest difference between the group where a low-fat diet (LFD) was consumed and the group where the Lactobacillus plantarum HAC03 strain and rutin (HR) were administered in combination. Even in view of the figures consisting of PCoA1 and PCoA3, it is possible to confirm that there is little difference between the group where the Lactobacillus plantarum HAC03 strain and rutin (HR) were administered in combination and the group where a low-fat diet (LFD) was consumed.

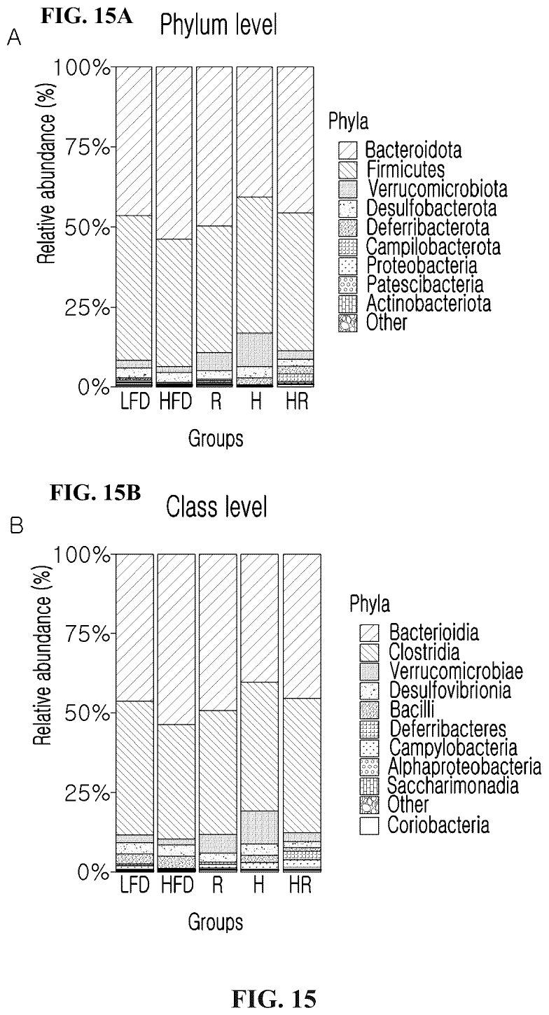

Additionally, FIG. 15 shows the results of observing the relative distribution of intestinal microorganisms at the Phylum (A) or Class (B) level.

### (3) Results of Relative Distribution of Intestinal Microorganisms

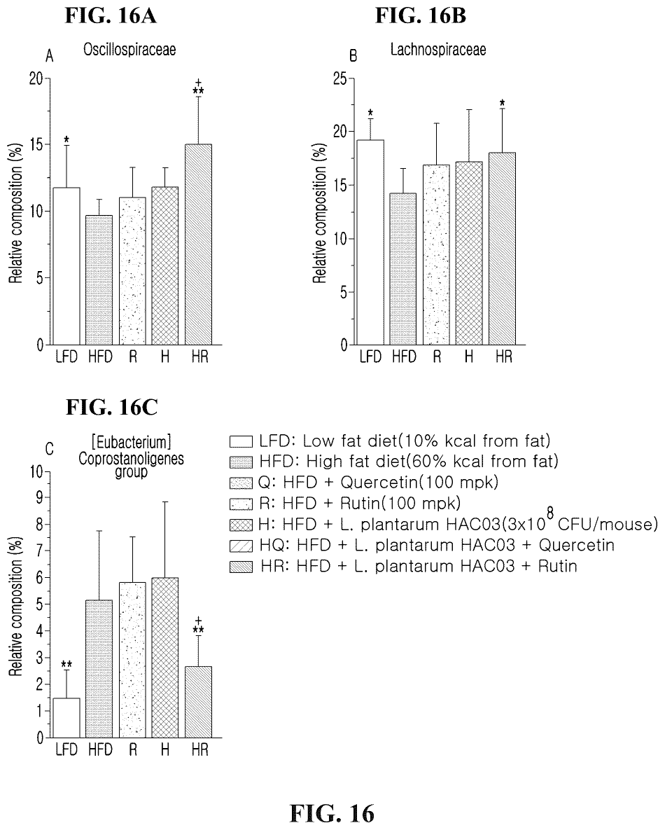

FIG. 16 analyzes the relative distribution of intestinal microorganisms, in which FIG. 16A shows the relative distribution of Oscillospiraceae, FIG. 16B shows the relative distribution of Lachnospiraceae, and FIG. 16C shows the relative distribution of Eubacterium Coprostanoligenes.

Oscillospiraceae is a bacterium that is significantly reduced in obese patients (Yang, Jingpeng, et al. “Oscillospira-a candidate for the next-generation probiotics.” Gut Microbes 13.1 (2021): 1987783). It was confirmed that compared to the group, in which only a high-fat diet (HFD) was consumed, the relative distribution of bacteria significantly increased in the group (HR) where the Lactobacillus plantarum HAC03 strain and rutin (HR) were administered in combination (FIG. 16A).

Lachnospiraceae is also a bacterium that is significantly reduced in obese patients (Lippert, Katrin, et al. “Gut microbiota dysbiosis associated with glucose metabolism disorders and the metabolic syndrome in older adults.” Beneficial microbes 8.4 (2017): 545-556.). It was confirmed that compared to the group, in which only a high-fat diet (HFD) was consumed, the relative distribution of bacteria significantly increased in the group (HR) where the Lactobacillus plantarum HAC03 strain and rutin (HR) were administered in combination (FIG. 16B).

In contrast, Eubacterium Coprostanoligenes is a bacterium abundant in patients with type 2 diabetes caused by obesity (Ahmad, Aftab, et al. “Analysis of gut microbiota of obese individuals with type 2 diabetes and healthy individuals.” PloS one 14.12 (2019): e0226372.).

It was confirmed that compared to the group, in which only a high-fat diet (HFD) was consumed, the relative distribution of bacteria significantly decreased in the group (HR) where the Lactobacillus plantarum HAC03 strain and rutin (HR) were administered in combination (FIG. 16C).

That is, it was confirmed that when the Lactobacillus plantarum HAC03 strain and rutin were administered in combination, an opposite tendency appeared contrary to what was shown in obese patients that the Eubacterium Coprostanoligenes microorganism significantly increased or the Oscillospiraceae and Lachnospiraceae microorganisms decreased.

Through this, it is possible to confirm that the combined administration of the Lactobacillus plantarum HAC03 strain and rutin can effectively treat or prevent obesity or insulin resistance syndrome.

### Deposit of Microorganisms

Depository Institution: Korea Research Institute of Bioscience and Biotechnology (KRIBB),

Biological Resource Center (KCTC) Accession Number: KCTC13242BP Accession Date: 2020 Dec. 28

What is claimed is:

1. A pharmaceutical composition for preventing or treating obesity or insulin resistance syndrome, comprising Lactobacillus plantarum HAC03 strain (Accession No: KCTC 13242BP) and rutin as active ingredients,
wherein the pharmaceutical composition reduces one or more selected from the group consisting of total cholesterol, triglycerides, low density lipoprotein cholesterol (LDL), alanine aminotransferase (ALT), and aspartate aminotransferase (AST), and
reduces blood sugar fluctuations, fasting blood sugar levels, and fasting insulin levels.

2. The pharmaceutical composition of claim 1, wherein the composition exhibits an anti-obesity effect by regulating the expression levels of one or more genes selected from the group consisting of fatty acid synthase (FAS), acetyl COA carboxylase (ACC), peroxisome proliferator-activated receptor γ (PPARγ), sterol regulatory element binding protein 1C (SREBP1C), peroxisome proliferator-activated receptor α (PPARα), peroxisome proliferator-activated receptor γ coactivator 1 α (PGC1α), carnitine palmitoyltransferase 1 (CPT1), acyl-CoA oxidase 1 (ACOX1), uncoupling protein 1 (UCP1), and PR/SET domain 16 (PRDM16), and
wherein the anti-obesity effect is at least one effect selected from the group consisting of inhibition of lipogenesis, increase in fat oxidation, and increase in body thermogenesis.

3. The pharmaceutical composition of claim 1, wherein the composition reduces insulin resistance by regulating the expression level of glucose-6-phosphate dehydrogenase (G6P) gene or phosphoenolpyruvate carboxykinase (PEPCK) gene.

4. A health functional food composition for preventing or treating obesity or insulin resistance syndrome, comprising Lactobacillus plantarum HAC03 strain (Accession No: KCTC 13242BP) and rutin as active ingredients,
wherein the pharmaceutical composition reduces one or more selected from the group consisting of total cholesterol, triglycerides, low density lipoprotein cholesterol (LDL), alanine aminotransferase (ALT), and aspartate aminotransferase (AST), and reduces blood sugar fluctuations, fasting blood sugar levels, and fasting insulin levels.

5. The health functional food composition of claim 4, wherein the composition exhibits an anti-obesity effect by regulating the expression levels of one or more genes selected from the group consisting of fatty acid synthase (FAS), acetyl CoA carboxylase (ACC), peroxisome proliferator-activated receptor γ (PPARγ), sterol regulatory element binding protein 1C (SREBP1C), peroxisome proliferator-activated receptor α (PPARα), peroxisome proliferator-activated receptor γ coactivator 1 α (PGC1α), carnitine palmitoyltransferase 1 (CPT1), acyl-CoA oxidase 1 (ACOX1), uncoupling protein 1 (UCP1), and PR/SET domain 16 (PRDM16), and
wherein the anti-obesity effect is at least one effect selected from the group consisting of inhibition of lipogenesis, increase in fat oxidation, and increase in body thermogenesis.

6. The health functional food composition of claim 4, wherein the composition reduces insulin resistance by regulating the expression level of glucose-6-phosphate dehydrogenase (G6P) gene or phosphoenolpyruvate carboxykinase (PEPCK) gene.


SEQUENCE LISTING (27 sequences)

SEQ ID NO: 1 - DNA, 1404 bp, unidentified
  source 1..1404: mol_type = genomic DNA
ctctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgtccgtcacaaccatgagagtttgtaacacccaaagtcggtggggtaacc

SEQ ID NO: 2 - DNA, 19 bp, unidentified
  source 1..19: mol_type = other DNA
ctggactcgctcatgggtg

SEQ ID NO: 3 - DNA, 21 bp, unidentified
  source 1..21: mol_type = other DNA
catttcctgaagtttccgcag

SEQ ID NO: 4 - DNA, 23 bp, unidentified
  source 1..23: mol_type = other DNA
tgacagactgatcgcagagaaag

SEQ ID NO: 5 - DNA, 18 bp, unidentified
  source 1..18: mol_type = other DNA
tggagagccccacacaca

SEQ ID NO: 6 - DNA, 17 bp, unidentified
  source 1..17: mol_type = other DNA
agtggagaccgcccagg

SEQ ID NO: 7 - DNA, 21 bp, unidentified
  source 1..21: mol_type = other DNA
gcagcaggttgtcttggatgt

SEQ ID NO: 8 - DNA, 21 bp, unidentified
  source 1..21: mol_type = other DNA
agcagcccctagaacaaacac

SEQ ID NO: 9 - DNA, 21 bp, unidentified
  source 1..21: mol_type = other DNA
cagcagtgagtctgccttgat

SEQ ID NO: 10 - DNA, 24 bp, unidentified
  source 1..24: mol_type = other DNA
gtacggtgtgtatgaagccatctt

SEQ ID NO: 11 - DNA, 18 bp, unidentified
  source 1..18: mol_type = other DNA
gccgtacgcgatcagcat

SEQ ID NO: 12 - DNA, 20 bp, unidentified
  source 1..20: mol_type = other DNA
cctgaagccgggagagaatg

SEQ ID NO: 13 - DNA, 20 bp, unidentified
  source 1..20: mol_type = other DNA
tagccagcagagactgtgga

SEQ ID NO: 14 - DNA, 19 bp, unidentified
  source 1..19: mol_type = other DNA
tgagtggcgtcctctttgg

SEQ ID NO: 15 - DNA, 21 bp, unidentified
  source 1..21: mol_type = other DNA
tcagcgagtagcgcatagtca

SEQ ID NO: 16 - DNA, 22 bp, unidentified
  source 1..22: mol_type = other DNA
gtgcagctcagagtctgtccaa

SEQ ID NO: 17 - DNA, 22 bp, unidentified
  source 1..22: mol_type = other DNA
tactgctgcgtctgaaaatcca

SEQ ID NO: 18 - DNA, 21 bp, unidentified
  source 1..21: mol_type = other DNA
ctttgcctcactcaggattgg

SEQ ID NO: 19 - DNA, 21 bp, unidentified
  source 1..21: mol_type = other DNA
actgccacacctccagtcatt

SEQ ID NO: 20 - DNA, 20 bp, unidentified
  source 1..20: mol_type = other DNA
gaagtcacaggaggacacgg

SEQ ID NO: 21 - DNA, 20 bp, unidentified
  source 1..20: mol_type = other DNA
ctcgctcctcaacacacctc

SEQ ID NO: 22 - DNA, 21 bp, unidentified
  source 1..21: mol_type = other DNA
cgactcgctatctccaagtga

SEQ ID NO: 23 - DNA, 20 bp, unidentified
  source 1..20: mol_type = other DNA
gttgaaccagtctccgacca

SEQ ID NO: 24 - DNA, 21 bp, unidentified
  source 1..21: mol_type = other DNA
ctgcataacggtctggacttc

SEQ ID NO: 25 - DNA, 19 bp, unidentified
  source 1..19: mol_type = other DNA
cagcaactgcccgtactcc

SEQ ID NO: 26 - DNA, 21 bp, unidentified
  source 1..21: mol_type = other DNA
ggcaccacacyttctacaatg

SEQ ID NO: 27 - DNA, 21 bp, unidentified
  source 1..21: mol_type = other DNA
ggggtgttgaaggtctcaaac